(19) United States
(12) Patent Application Publication
(10) Pub. No.: US 2026/0179755 A1
(43) Pub. Date: Jun. 25, 2026

(54) SYSTEM AND METHOD OF WOUND ASSESSMENT

(71) Applicant: Sreehita HAJEEBU, Burlington, NJ (US)

(72) Inventor: Sreehita HAJEEBU, Burlington, NJ (US)

(21) Appl. No.: 19/127,595

(22) PCT Filed: Nov. 6, 2023
(86) PCT No.: PCT/US2023/036869

§ 371 Date: May 6, 2025

Related U.S. Application Data
(60) Provisional application No. 63/423,011, filed on Nov. 6, 2022.

(51) Int. Cl. G16H 30/20; A61B 5/00; A61M 1/00

(52) U.S. Cl. CPC G16H 30/20; A61B 5/0077; A61M 1/95

(57) ABSTRACT

Assessing a wound by capturing, via a mobile device proximate the person (or animal) suffering from the wound, still and/or moving imagery of the wound as well as voice dictation/annotation, processing the captured imagery and annotations at a remote processing engine to automatically create a respective three-dimensional (3D) model of the wound, determine wound measurements using vertices and edges defined by the 3D model, determine wound characteristics using the wound measurements, and determine wound classification using the wound characteristics and audio annotation. Whereupon a classification-specific wound care treatment may be retrieved from a wound care knowledge base and transmitted to the mobile device.

### CROSS REFERENCE TO RELATED APPLICATION

This application claims the benefit of the filing date of U.S. Provisional Patent Application No. 63/423,011 filed Nov. 6, 2022, the disclosure of which is incorporated herein by reference in its entirety.

### FIELD OF THE DISCLOSURE

The present disclosure generally relates to wound assessment and, more particularly, to a system and method for capturing and remotely assessing wound imagery and related audio annotations.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Introduction. Wounds and the process of wound healing represent intrinsic aspects of human existence. Wounds manifest when the integrity of the skin is compromised, either due to injuries or because of underlying medical conditions. While certain wounds can be effectively managed at home using basic first-aid techniques, others necessitate professional medical intervention. These include pressure injuries, wounds associated with diabetes, moisture-related wounds, traumatic injuries, and post-surgical wounds, among others.

Wound Care. Wound care is an indispensable component of the wound life cycle and overall wound management. It encompasses a multifaceted approach that involves the detection, classification, diagnosis, consideration of factors influencing the healing process, and the strategic application of appropriate measures to facilitate the treatment and healing of wounds.

Essential Aspects. As straightforward as these concepts may appear, two pivotal aspects stand out as the linchpins for providing appropriate wound care and recovery: (a) accurately identifying the underlying cause of the wound or health issue, and (b) determining the most suitable, patient-specific wound care approach.

Notable Challenges in Wound Care. The landscape of wound care is riddled with challenges, each of which presents intricate technical dilemmas, including a lack of unified and standardized practices, a limited number of specialized professionals, high costs, and patient burden.

Lack of Integrated and Standardized Practices: A lack of unified and standardized practices within the realm of wound care leads to inefficiencies and disjointed care transitions. Wounds, while common, are not classified as actual medical conditions. Additionally, there is no distinct specialization in wound management. Consequently, patients find themselves receiving care from a multitude of practitioners with varying skill sets and areas of expertise, including clinicians, nurses, general practitioners, surgeons, and physical therapists. This diversity in care providers often results in varying treatment approaches based on individual experiences and perspectives. Although some evidence-based guidelines exist, there is a dearth of standard protocols and guidelines specifying the most effective treatment options and their timing. This deficiency in coordination results in a wound care landscape that heavily relies on personnel-driven, knowledge-centric, and expertise-based approaches.

Limited Specialized Professionals: Another pressing challenge is the scarcity of specialized professionals, skilled care providers, and trained nurses dedicated to wound care. Even when such professionals are available, the high risk of recruitment by competing healthcare providers places immense strain on the already delicate wound care ecosystem.

High Costs: The cost of wound care remains notably high, particularly within inpatient programs. The predominant approach to wound care is often reactive and rooted in traditional practices. The overall expenditure is significantly attributed to nursing hours and hospitalization costs, accounting for approximately 80% of the total expenses.

Burden on Patients: Patients with wounds endure various burdens on physiological, psychological, social, and financial fronts, resulting in suboptimal experiences. Many patients, especially those facing transportation challenges, such as the elderly or individuals residing in rural areas, prefer receiving care within the comfort of their homes. The unavailability of such an option can lead to missed or cancelled appointments, increasing the risk of infections, deteriorations, and potentially, amputations.

Improvements are desired.

### SUMMARY

Various deficiencies in the prior art are addressed by systems, methods, architectures, mechanisms, and apparatus for assessing a wound by capturing, via a mobile device proximate the person (or animal) suffering from the wound, still and/or moving imagery of the wound as well as voice dictation/annotation, processing the captured imagery and annotations at a remote processing engine to automatically create a respective three-dimensional (3D) model of the wound, determine wound measurements using vertices and edges defined by the 3D model, determine wound characteristics using the wound measurements, and determine wound classification using the wound characteristics and audio annotation. Whereupon a classification-specific wound care treatment may be retrieved from a wound care knowledge base and transmitted to the mobile device.

A method of wound assessment according to one embodiment comprises: at a mobile device, capturing wound related imagery and respective audio annotation, and transmitting the wound related imagery and respective audio annotation towards a remote processing engine; at the remote processing engine: processing received wound related imagery to create a respective three-dimensional (3D) model of the wound; determining wound measurements using vertices and edges defined by the 3D model of the wound; determining wound characteristics using the wound measurements; determining wound classification using the wound characteristics and audio annotation; retrieving a wound care treatment for the wound classification from a knowledge base; and transmitting the wound care treatment toward the mobile device.

Additional objects, advantages, and novel features of the invention will be set forth in part in the description which follows and will become apparent to those skilled in the art upon examination of the following or may be learned by practice of the invention. The objects and advantages of the invention may be realized and attained by means of the instrumentalities and combinations particularly pointed out in the appended claims.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, explain the principles of the present invention.

FIG. 1 depicts a high-level block diagram of a wound assessment system according to various embodiments;

FIG. 2 depicts a high-level block diagram illustrating various functions and interactions between components of a wound assessment system according to an embodiment;

FIGS. 3A-3F depict various mobile device user interface images;

FIG. 4 depicts an exemplary portal dashboard screen;

FIGS. 5A-5C depict exemplary wound view user interface screens;

FIGS. 6A-6D depict exemplary wound measurement user interface screens;

FIGS. 7-9 depict flow diagrams illustrating methods according to various embodiments;

FIG. 10 depicts a high-level block diagram illustrating various functions and interactions between components of a wound assessment system according to an embodiment; and

FIG. 11 depicts a high-level block diagram illustrating an exemplary implementation of a wound assessment system according to an embodiment.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for pedagogical purposes to aid the reader in understanding the principles of the invention and the concepts contributed by the inventor(s) to furthering the art and are to be construed as being without limitation to such specifically recited examples and conditions. Additionally, the term, “or” as used herein, refers to a non-exclusive or, unless otherwise indicated (e.g., “or else” or “or in the alternative”). Also, the various embodiments described herein are not necessarily mutually exclusive, as some embodiments may be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

Various embodiments contemplate a system (denoted herein as “Cogniwound” of the “Cogniwound system”) generally comprising three primary components; namely, mobile devices/apps, a remote processing engine (RPE), and a web portal. The mobile devices/apps are configured for collecting wound related data such as wound photos and videos, along with comprehensive wound characteristics such as via audio annotations from a first responder or other on-the-scene personnel attending to a patient presenting with one or more wounds. The wound data is then securely transmitted to the remote processing engine (RPE) for analysis, such a wound identification, characterization, and so on. The processing engine interfaces with various functional modules to process the wound data, extract wound measurements therefrom, characterize/classify/diagnose the wound, and generally provide insight as to the wound and relevant treatments for the wound (e.g., generating predictive and prescriptive intelligence based on the wound data). The web portal serves as a consolidated interface that utilizes wound characterization, classification, diagnosis, and other wound insight data to support wound care decision functions and to provide a resource for clinical users to access, search, and refer to. The various embodiments may further allow allows for the fine-tuning of system-generated treatment recommendations as per the specific requirements of the patient.

Various embodiments provide several key elements or components working together to perform the various functions, such as a mobile application, a cloud-based processing engine, and a web portal. Various embodiments are directed to a system, apparats, and related methodologies designed to confront the challenges that afflict wound care such as described herein. Various embodiments contemplate system, apparats, and related methodologies benefiting from some or all of Artificial Intelligence (AI), Machine Learning (ML), Augmented Reality (AR), Photogrammetry, and Statistical and Mathematical principles as will be discussed in more detail below.

The Cogniwound platform provides, in various embodiments, a diverse range of advanced features, including non-contact, precise wound measurement, voice-interactive wound dictation, 3D wound modeling, custom measurements on 3D wound models, and a self-evolving, context-aware wound healing prediction and prescription system. It offers a comprehensive suite of services and functionalities that enable clinicians to deliver superlative wound care.

### a High-Level View of Cogniwound

Cogniwound High-Level Features: Wound Capture: This feature facilitates the acquisition of wound images, videos, and detailed documentation; Wound 3D Model Generation and Rendering: Cogniwound automates the creation and visualization of 3D wound models; Wound Detection, Classification, Measurement, and Analysis: These processes are performed automatically, streamlining wound assessment and analysis; Wound Predictive and Prescriptive Intelligence: Cogniwound incorporates advanced algorithms to offer predictive insights into wound prognosis and prescriptive recommendations for treatment. This functionality continually evolves through learning from real-world wound treatments; Integration and Seamless Data Exchange with EMRs/Other Clinical Systems: Cogniwound seamlessly interfaces with Electronic Medical Records (EMRs) and other clinical systems, enabling efficient data exchange; Standalone/Integrated Mode: Cogniwound can be used as a standalone application or integrated into existing healthcare systems, offering flexibility to suit the provider's needs; Offline/Online Mode: The platform supports both offline and online modes, ensuring that healthcare professionals can access its capabilities irrespective of their connectivity status.

Central to Cogniwound is its wound care knowledge base. This repository aggregates wound care protocols and procedures from diverse sources, encompassing structured, unstructured, and semi-structured data. It acts as a unifying force, standardizing the wealth of information. This knowledge base is enriched by integrating patient data, wound data, and information from EMRs and other healthcare systems. By merging these data sources, Cogniwound is equipped to conduct comprehensive wound diagnosis and provide tailored recommendations for wound treatment, directly benefiting patients. What sets this system apart is its continuous self-improvement. As it learns from the real-world applications of wound treatments, Cogniwound evolves, enhancing its knowledge base to consistently provide higher levels of service.

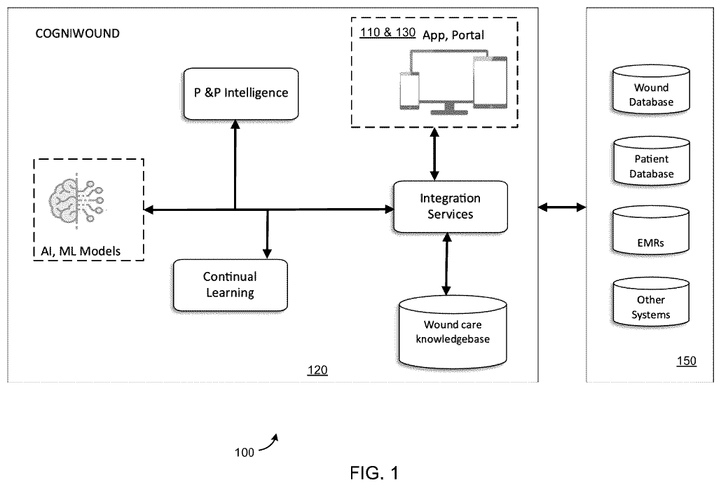

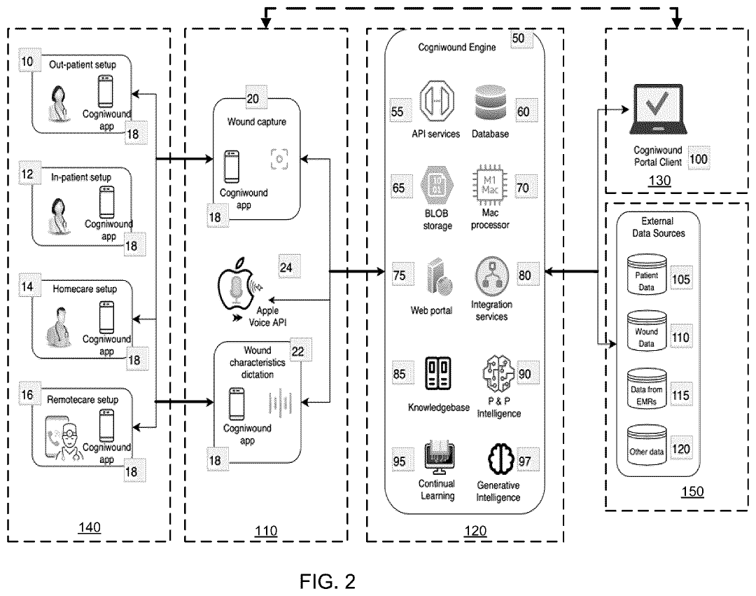

FIG. 1 depicts a high-level block diagram of a wound assessment system according to various embodiments, and FIG. 2 depicts a high-level block diagram illustrating various functions and interactions between components of the wound assessment system of FIG. 1. Many modifications to the embodiments depicted in FIGS. 1-2 may be made, as will be described in more detail below.

Generally speaking, the wound assessment system 100 of FIGS. 1-2 comprises one or more mobile devices 110 in communication with a remote processing engine (RPE) 120 (and optionally with a web portal 130), the web portal 130 in communication with the RPE 120 and external data sources 150, and a plurality of setup mechanisms 140 in communication with at the one or more mobile devices 110 to provide setup information thereto.

### Cogniwound Mobile Device/App 110

The mobile devices 110 may comprise mobile phones, laptops, or special purpose mobile telecommunications/computing devices configured to execute applications or apps so as to perform the various functions described herein.

In particular, the mobile devices 110 are configured to execute a mobile app that performs a number of functions, including: capturing still or moving images of a wound; capturing audio annotations pertaining to the wound, such as by a first responder interacting with the mobile device while examining a patient presenting with a wound; and/or perform various other functions as described herein.

Various embodiments contemplate that the mobile device/app 110 includes a functional module configured to determine wound metadata (gravity, depth, and alpha mask) at the time of wound 2D image capture.

In various embodiments, the mobile app may leverage one or more advanced technological components such as those rooted in Artificial Intelligence (AI), Augmented Reality (AR), Photogrammetry, Statistical and Mathematical principles to perform a various range of intricate functions. For example, various embodiments contemplate that Augmented Reality capable smartphones are configured to perform no-touch wound measurements using advanced algorithms built into the AR system.

In various embodiments, the mobile device/app provides optimized image and video capture. That is, the mobile app is engineered to capture an optimal number of wound images or videos. This feature ensures that a comprehensive visual record of the wound is obtained, facilitating a more precise understanding of the wound's condition.

In various embodiments, the mobile device/app provides camera positioning and control instructions/tracking. That is, an autonomous camera positioning system is integrated therein, enabling the mobile camera to adaptively capture wound images or videos from multiple angles and viewpoints. This dynamic approach provides a 360-degree view of the wound, which is particularly valuable in assessing wounds with complex topography.

In various embodiments, the mobile device/app provides voice-interactive dictation and audio capture. That is, the mobile device/app includes a voice-interactive dictation component that permits the user to verbally record wound characteristics. This feature enhances the efficiency of data input, reducing the potential for errors in manual documentation.

In various embodiments, the mobile device/app provides wound image metadata. That is, the mobile device/app is equipped to determine critical wound image metadata, including factors such as gravity, depth, and alpha mask. These metadata components are fundamental for generating detailed 3D models of the wound.

In various embodiments, the mobile device/app provides 3D wound model Rendering. That is, the mobile device/app provides an ability to render 3D models of the wound directly within the application. This capability is a result of the gathered metadata and images, allowing for a holistic, three-dimensional representation of the wound. This 3D modelling enhances the diagnostic and visualization aspects of wound assessment, which are crucial in formulating effective treatment plans.

In various embodiments, the mobile device/app provides both online and offline functionality. That is, the mobile device/app operates in online and offline modes, as well as seamlessly switching between both modes. In an online environment, the device/app connects to a stable Wi-Fi network or cellular network, enabling the real-time transmission of wound data to a cloud-based repository. This ensures the prompt availability of data to the broader wound care ecosystem. In cases where network connectivity is compromised, the device/app seamlessly transitions to offline mode. In this configuration, wound data is securely stored locally on the device, awaiting synchronization with the cloud-based system once a reliable network connection is re-established. The voice-interactive dictation component is exclusively functional in the online mode, further illustrating the adaptability of the app to various operational scenarios.

In general, the Cogniwound mobile app is configured to perform various functions, including capturing wound details; wound characteristics annotation via voice-interactive dictation; displaying wound 3d models; performing various online and offline functions as described herein; and/or performing other functions as described herein.

Capturing Wound Details: The Cogniwound mobile app serves as the primary data collection interface, facilitating the capture of comprehensive wound details. This includes wound photos and/or wound videos, which are essential for a thorough assessment.

Wound Characteristics Annotation via Voice-Interactive Dictation: The app features a voice-interactive dictation component, enabling the precise annotation of wound characteristics through voice commands. This significantly streamlines data entry and minimizes the potential for errors.

Displaying Wound 3D Models: One of the groundbreaking features of the app is its capability to display wound 3D models. These 3D models offer an advanced and holistic view of the wounds, greatly enhancing the visualization and analysis of wound conditions. It is noted that the mobile device generally captures 2D still or moving imagery of a wound, which imagery is processed by the RPE 120 to generate 3D imagery, which may then be provided to the mobile device 110 and displayed using 3D display techniques, such as to enable a first responder to manipulate the image of the display device in a simulated 3D manner.

Online and Offline Functionality: The mobile app is engineered to function seamlessly in both online and offline modes. In the online mode, it connects to a reliable Wi-Fi or mobile cellular network, allowing for real-time data transmission. In the offline mode, data captures are stored locally on the device and can be transmitted to a cloud-based setup once a reliable network connection becomes available.

### Cogniwound Remote Processing Engine (RPE) 120

The remote processing engine (RPE) 120 may comprise one or more servers, functions instantiated at a data center via compute and memory resources, or any type of computing machinery configured to perform the various functions described herein.

In various embodiments, the RPE includes an AI and ML-powered processing engine serving as a technological backbone for numerous functions as described herein.

In various embodiments, the RPE provides wound detection and classification. That is, responsive to wound data received from the mobile device/app, the RPE provides automatic detection and classification of wounds within images or videos. The RPE may employ advanced pattern recognition algorithms to categorize wounds based on their characteristics, such as size, shape, and depth.

In various embodiments, the RPE provides automatic wound measurements. That is, the RPE is configured for automatically extracting precise measurements of wounds, providing crucial quantitative data that is indispensable in assessing wound progression and recovery.

In various embodiments, the RPE provides automatic characterization of wounds and surrounding skin. That is, the RPE analyzes wound measurements, imagery, voice annotations and so on to characterize the wound and the skin surrounding the wound. The RPE may use data analytics and machine learning algorithms to identify key attributes that aid in understanding the wound environment.

In various embodiments, the RPE provides 3D model generation. That is, the RPE generates 3D models of wounds based on received 2D images and/or videos. This process involves a complex reconstruction of the wound in three dimensions, providing a comprehensive visualization that aids in diagnosis and treatment planning.

In various embodiments, the RPE provides identification of representative images. That is, the RPE selects one or more representative wound images from a set of wound photos or videos as being most informative or characteristic of the wound for use in the wound documentation process, thereby ensuring that the most informative image is utilized for analysis and reporting.

In various embodiments, the RPE incorporates AI and ML technologies to achieve the standardization of wound care procedures and protocols to ensure that wound care is consistently efficient and patient-focused. Moreover, such standardization equips clinicians with the tools and resources needed to enhance standardized wound care procedures and protocols.

In general, the Cogniwound Remote Processing Engine (RPE) is configured to perform various functions, including: conversion of 2d wound media to 3d models; perform wound measurements relative to 3d models; automatic detection of wound characteristics; automated detection, classification, and diagnosis of wounds; integration with external systems; provide for and/or manage a wound care knowledge base, provide for and/or manage wound care predictive and prescriptive intelligence; provide for and/or manage continuous learning and knowledge base improvement; provide for and/or manage generative ai for wound images and characteristics; and/or perform various other functions as described herein.

Conversion of 2D Wound Media to 3D Models: The core function of the Cogniwound engine is to convert 2D wound photos and/or wound videos into precise 3D models. This transformation greatly enhances the depth and accuracy of wound assessment.

Wound Measurements Relative to 3D Models: The engine excels at determining wound measurements relative to vertices and edges on 3D models. This capability is pivotal in capturing precise data for wound assessment and monitoring.

Automatic Detection of Wound Characteristics: Advanced algorithms are deployed to automatically detect wound characteristics, providing a comprehensive understanding of the wound's attributes.

Automated Detection, Classification, and Diagnosis of Wounds: The engine is adept at automatically detecting, classifying, and diagnosing wounds. This automation streamlines the initial diagnostic process, eliminating guesswork and improving accuracy.

Integration with External Systems: The engine is designed to seamlessly communicate with other healthcare systems, facilitating the exchange of patient, wound, and treatment plan-related data. This interoperability enhances the overall efficiency of the wound care process.

Wound Care Knowledge Base: The engine is responsible for constructing and maintaining a comprehensive wound care knowledge base. This repository draws from a multitude of sources, including wound characteristics, patient attributes, patient-wound history, standardized wound care procedures and protocols, and actual treatment plans administered.

Wound Care Predictive and Prescriptive Intelligence: The engine is equipped to provide predictive and prescriptive intelligence in the treatment of wounds. This intelligence is derived from extensive data analysis, eliminating uncertainty and guesswork in the treatment process.

Continuous Learning and Knowledge Base Improvement: The engine operates on a continuous learning model. It learns from actual wound care treatments administered, leveraging this knowledge to enhance its predictive and prescriptive capabilities. This ongoing learning process results in a continually improved wound care knowledge base.

Generative AI for Wound Images and Characteristics: The engine leverages generative AI to create synthetic wound images and characteristics. This not only enhances the quality of data but also offers additional resources for training and analysis.

### Cogniwound Portal 130

The portal 130 may comprise one or more servers, functions instantiated at a data center via compute and memory resources, or any type of computing machinery configured to perform the various functions described herein. The portal 130 is configured to perform various functions, including some or all of: rendering wound 3d models and measurement; managing predictive and prescriptive intelligence; managing access to wound care knowledge base; managing refinement of treatment plans; and/or perform various other functions as described herein.

Rendering Wound 3D Models and Measurement: The portal serves as the interface for rendering detailed 3D models of wounds. Moreover, it allows for precise measurements to be taken on these 3D models, offering an advanced level of analysis that is instrumental in wound care.

Predictive and Prescriptive Intelligence: The portal is equipped to provide predictive and prescriptive intelligence on the treatment of wounds. This intelligence is underpinned by advanced statistical and mathematical models, offering actionable recommendations for wound healing.

Access to Wound Care Knowledge Base: The portal facilitates access to the wound care knowledge base constructed and maintained by the engine. This knowledge base provides valuable references and resources for healthcare professionals.

Refinement of Treatment Plans: An important feature of the portal is its capacity to refine treatment plans. It offers a platform for clinicians to fine-tune and customize treatment plans based on the specific needs of patients.

Referring to FIG. 2, it can be seen that a mobile app (18) plays a central role in the process of capturing, analyzing, and documenting wounds. The mobile app 18 effectively harnesses an array of advanced technological concepts, including Artificial Intelligence (AI), Photogrammetry, Statistical, and Mathematical principles, to perform its diverse functions.

The key features and capabilities of the mobile app 18 include Capture of 2D Wound Media; Metadata Collection for 3D Model Conversion (20); Wound Characteristics Annotation through Voice-Interactive Dictation (22); Secure Data Transmission via API Services (55); Storage in a cloud database/datacenter such as AWS or Azure Cloud Database (60); and/or other functions as described herein.

Capture of 2D Wound Media (18): The primary and foundational function of the mobile app is to capture 2D wound photos and/or wound videos. These visual representations are integral for a comprehensive understanding of the wound's condition and progress.

Metadata Collection for 3D Model Conversion (20): Concurrently with the capture of 2D wound media, the mobile app also gathers crucial metadata. This metadata is indispensable for the subsequent conversion of 2D objects into detailed 3D models.

Wound Characteristics Annotation through Voice-Interactive Dictation (22): Within the mobile app (18), a voice-interactive dictation module (22) is incorporated. This innovative feature empowers healthcare professionals to annotate wound characteristics with precision, using voice commands. This approach significantly enhances the efficiency of data entry while minimizing the potential for errors.

Secure Data Transmission via API Services (55): Ensuring the utmost data security and integrity, the mobile app employs API services (55) for the secure transmission of captured wound media and associated characteristics. This robust and secure data transfer mechanism safeguards the confidentiality of patient information.

Storage in Azure Cloud Database (60): The transmitted wound photos and/or wound videos, along with their associated metadata and annotated characteristics, find a secure and accessible home in a database (60) hosted on the Azure cloud platform. Azure's sophisticated infrastructure ensures not only the confidentiality of data but also scalability and compliance with the highest standards of data security.

As discussed in more detail below, there are generally four user roles contemplated with respect to different aspects of the embodiments; namely, super admin, administrator, doctor, and nurse (or first responder).

The super admin role may be specific to Cogniwound support team. This role will be used to create one or more administrator accounts (admin users) for a client.

The admin users of a client will be able to add users and assign doctor or nurse or first responder roles.

The users with doctor or nurse or first responder roles will be able to ‘submit’ patient visit records (i.e., they will be generally using the mobile device and related app).

User Interaction with the Cogniwound Mobile App

The Cogniwound mobile app 18 is designed to ensure a user-friendly and efficient experience for healthcare professionals. Various user interactions are contemplated, including the following (with reference to user interface screens where appropriate).

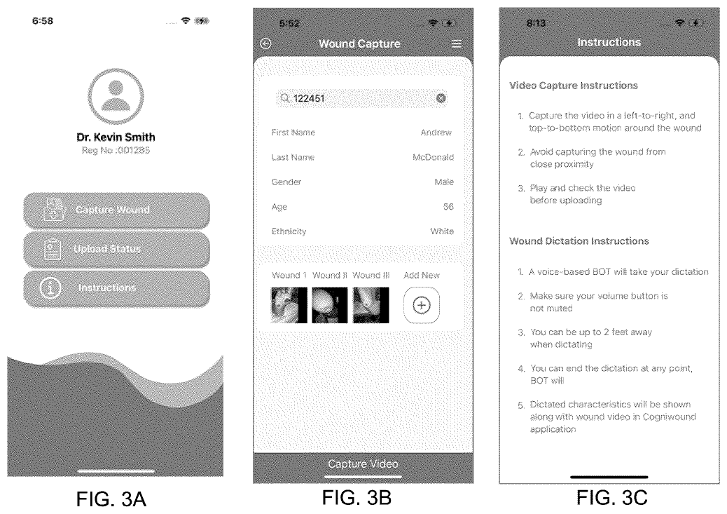

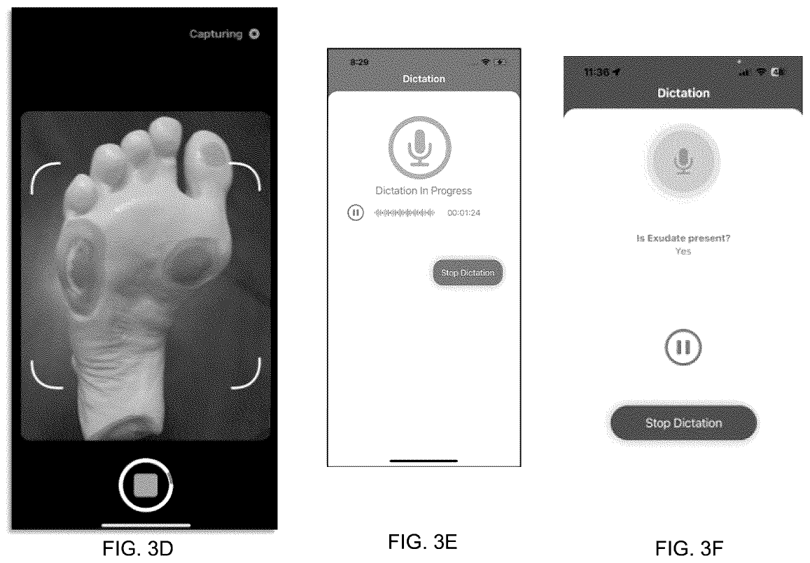

FIG. 3A depicts a mobile device user interface screen or image comprising an Initial Menu Screen with Three Options. Specifically, in response to use interaction with the mobile device 110 indicative of a desire to access the Cogniwound app, the mobile device user interface image of FIG. 3A is displayed and the Cogniwound app is invoked or otherwise executed, such that the user may then select via respective displayed icons one of three options; namely, (1) capture wound; (2) upload status; and (3) instructions.

### Capture Wound

User interaction indicative of selecting the Capture Wound icon invokes the various processes enabling the user to capture initial wound data (e.g., imagery, voice annotations, and the like) with the mobile device, including displaying the mobile device user interface image of FIG. 3B, which enables the user to Search Existing Patient ID (i.e., search for an existing patient's unique ID so as to streamline the data entry process), Enter Patient Details (e.g., for new patients, users can enter essential patient information, including the first name, last name, gender, age, and ethnicity), and add new still (pictures) or moving images (video) of the wound, voice annotations of the captured images, and so on.

User interaction indicative of selecting the “add new” wound icon invokes the various processes enabling the user to capture new wound imagery, including displaying the mobile device user interface image of FIG. 3C, which provides interactive instructions for capturing video, capturing pictures, and capturing dictation (voice annotations) to be associated with the capture pictures/video. These instructions guide the user through the process and ensure a comprehensive data collection approach (see FIGS. 3D-3F).

For example, the user may be instructed to capture a 20 to 45-second video of the wound from an approximate distance of two feet from the wound, to keep the camera steady in front of the wound, and so on. In various embodiments, the app is programmed to automatically capture different angles of the wound, ensuring a comprehensive visual record. That is, the user is instructed to move the camera of the mobile device in a particular manner or to particular vantage points with respect to the wound, whereupon the image is automatically captured by the app (or manually by the user).

Wound Characteristics Dictation: Following or during video capture, the user is prompted to dictate wound characteristics into the app. The app may display to the user various questions related to the wound's condition, such as “Is exudate present?” or “Exudate amount?” Users are required to answer each question and can say “next” to proceed to the next inquiry. The user may be given the option to pause the dictation at any point, thereby enhancing flexibility and convenience.

The dictation model is particularly valuable as it allows wound care providers to input critical information without the need for time-consuming processes like sanitizing hands or removing gloves. This approach streamlines the visit, making it more time-efficient.

Automatic Video Stop: The app employs advanced algorithms to automatically stop the video capture when it has accumulated sufficient data to render an accurate 3D model. This automated stop ensures efficiency and accuracy in the data collection process.

### Upload Status

User interaction indicative of selecting the Upload Status icon invokes the various processes enabling the user to upload status information pertaining to a patient's wound (e.g., after initial data is captured and uploaded), providing thereby crucial updates on the wound's progress. Within an upload status mobile device user interface screen, pictures and videos being uploaded to the Cogniwound cloud or remote processing engine 120 may be displayed. These may be categorized by their status, which can be either “in-progress” or “failed.” Videos listed under “in-progress” are those currently undergoing the upload process, while those categorized as “failed” are instances where video uploads were unsuccessful due to poor network connections. Users can access this feature to view and manage “failed” videos stored on their phone, with the option to initiate a reupload by simply clicking the designated button for each video.

3D Model Rendering: After approximately 5 to 10 minutes or processing at the RPE 120, the 3D model of the wound is fully rendered and may be downloaded to the mobile device 110 if desired (e.g., to enable detailed visual examination of the wound by first responders, doctors, etc.). The 3D model offers a detailed and comprehensive representation of the wound's structure.

Patient Dashboard for Tracking: To track the healing progress of the wound, users can access the patient dashboard at a later time. This dashboard provides updated trends in wound healing based on the measurements taken during previous visits.

### Instructions

User interaction indicative of selecting the Instructions icon invokes the various processes by which a user is provided access to comprehensive instructions and guidelines (stored at the mobile device or stored remotely), including displaying the mobile device user interface image of FIG. ??, which enables the user to access instruction information, thereby ensuring that the user properly uses the Cogniwound app and related functions.

User Interaction with the Cogniwound Portal

The Cogniwound Portal 130 provides healthcare professionals with intuitive, real-time access to comprehensive wound data, 3D models, and predictive intelligence, enhancing their ability to deliver precise and timely wound care. The user-friendly design ensures efficient navigation and data input, promoting optimal care delivery.

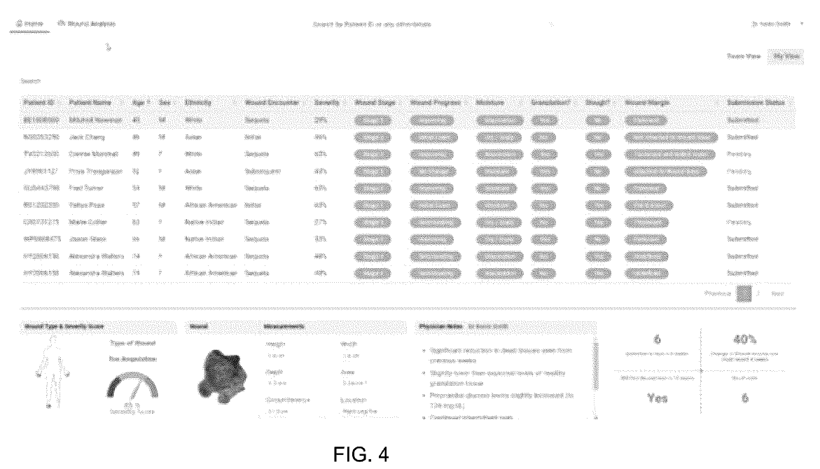

FIG. 4 depicts an exemplary portal dashboard screen. The portal dashboard screen in Cogniwound offers a centralized view of multiple patient wound details, including real-time status updates. Healthcare providers can conveniently access patient data, and with just a click, navigate to detailed wound views for comprehensive assessment and precise treatment planning. This feature enhances care efficiency and ensures that timely interventions are made for improved patient outcomes.

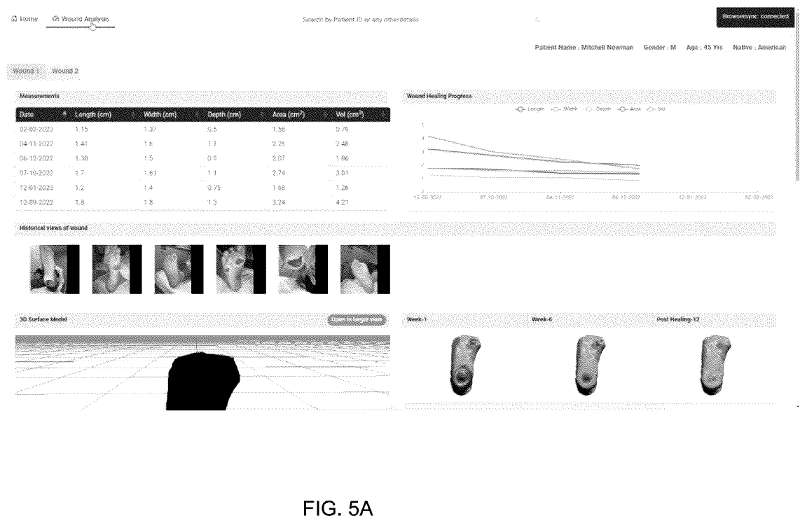

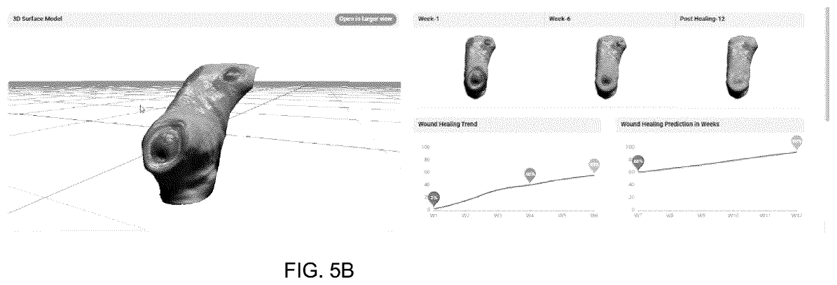

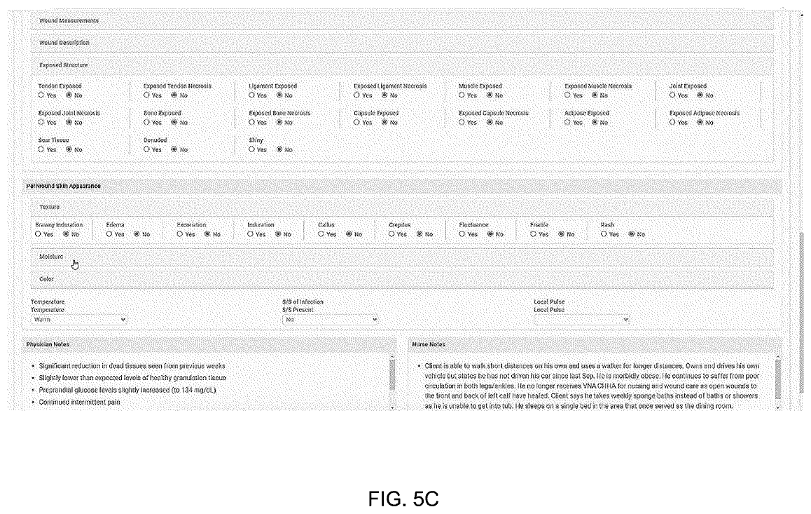

FIGS. 5A-5C depict exemplary wound view user interface screens. Cogniwound's Wound View Screen empowers healthcare providers with a comprehensive, patient-centered approach to wound care. Multiple patient wounds are efficiently organized as tabs, allowing clinicians to monitor wound healing progression, review historical wound measurements per visit, and access predictive and prescriptive intelligence for informed decision-making. The screen offers seamless wound characteristics documentation, where dictated information is readily available for review and editing. Clinicians can also add personalized notes, and the system provides evidence-based treatment recommendations, ensuring precision and effectiveness in patient care.

These screens are useful and provide a highly detailed platform tailored for managing patient wounds across a spectrum of healthcare contexts. It acts as the central hub for clinicians, providing them with a structured and comprehensive view of patients' wound histories and their ongoing healing journeys. Within the outpatient care domain, where clinicians face a multitude of patients with diverse wound types, this screen offers a streamlined process. It allows each patient's wound records to be displayed as separate tabs, ensuring that clinicians can swiftly navigate through various cases without the risk of confusion. This strategic organization minimizes errors and enhances the overall efficiency of wound assessments.

In inpatient care settings, such as hospitals and long-term care facilities, where the patients may have chronic conditions like pressure ulcers or complex surgical wounds, the Wound View Screen takes on a vital role. It serves as a digital repository of patient-specific data, presenting a historical perspective on wound measurements, healing progress, and the impact of various interventions. For instance, healthcare providers can meticulously observe how changes in treatment plans have influenced wound healing over time, thereby guiding them in making informed decisions about adjusting care strategies. This feature is especially critical for patients with diabetic foot ulcers, where a detailed understanding of wound healing patterns can be a key to delivering effective care.

The screen is also equipped with advanced predictive and prescriptive intelligence capabilities, benefiting healthcare providers in nursing homes and similar care facilities. Residents with long-standing wounds often require personalized and dynamic care. Cogniwound's AI-driven tools analyze patient data and wound characteristics, leveraging historical trends to formulate tailored treatment recommendations. This proactive aspect significantly elevates the likelihood of achieving positive patient outcomes, especially in cases where speedy wound healing is of paramount importance.

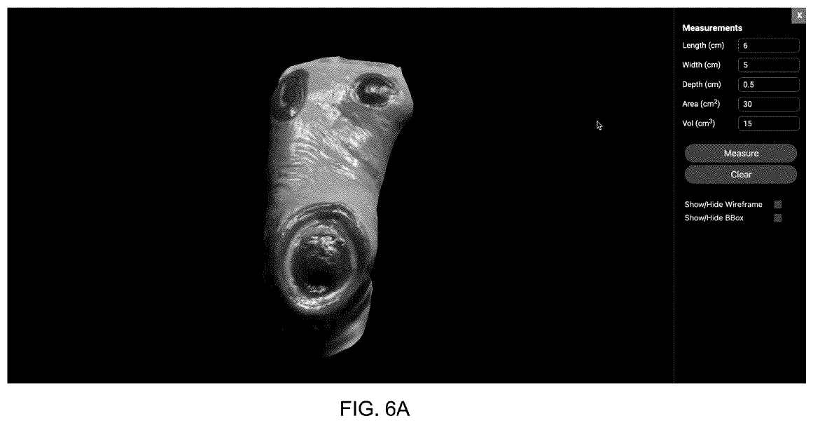

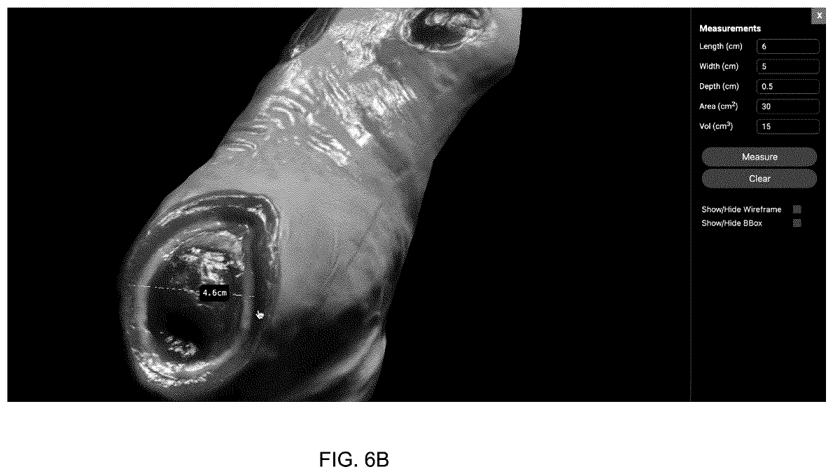

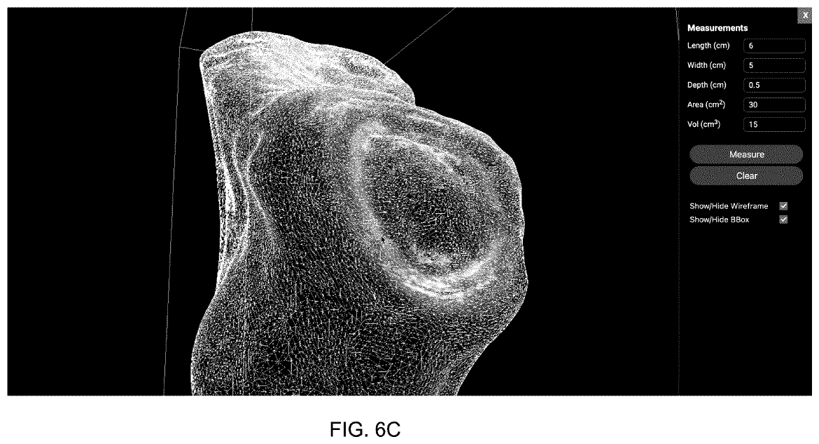

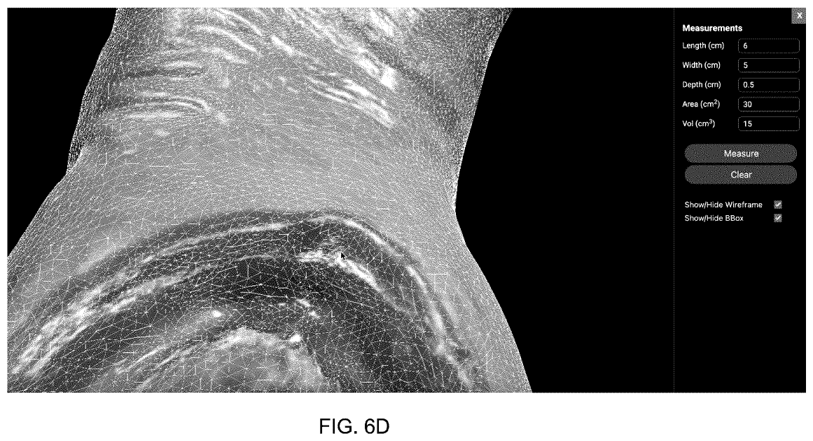

FIGS. 6A-6D depict exemplary wound measurement user interface screens: The Wound Measurement Screen in Cogniwound is a highly precise and efficient tool designed to simplify the process of wound measurement for healthcare professionals across various medical contexts. It offers an intuitive and non-creative solution by leveraging advanced technology to calculate key wound dimensions—height, width, depth, area, and volume. The system streamlines the measurement process to ensure that clinicians can focus on patient care while relying on Cogniwound's cutting-edge capabilities to provide measurements with an impressive accuracy rate of 99.99%.

In the clinical environment, precise wound measurements are crucial for diagnosis and effective treatment planning. Cogniwound's Wound Measurement Screen ensures that these measurements can be easily obtained with minimal effort. This non-creative solution represents a significant advancement in wound care, improving the accuracy and consistency of wound documentation across the board and contributing to better patient outcomes.

### Use Cases

Cogniwound's applications are versatile and cater to multiple healthcare settings. In outpatient care, clinicians efficiently capture wound data using voice-interaction, while inpatient care benefits from standardized and real-time wound documentation. The software enhances home and remote care by allowing detailed wound assessment, ensuring patients receive high-quality care in various scenarios.

Outpatient Care (10): In outpatient settings, healthcare professionals, including nurses and clinicians, utilize the Cogniwound mobile app during scheduled patient visits. They employ the app for several purposes, including (1) Capturing high-quality wound photos and videos with ease (thereby ensuring that a comprehensive visual record of the wound is maintained); and (2) Dictating wound characteristics through voice-interaction (thereby allowing for efficient and hands-free data entry. For instance, when assessing a diabetic patient with a chronic foot ulcer, clinicians use the app to quickly document wound characteristics like “exudate amount” and “presence of granulation tissue.” This integrated approach streamlines the wound assessment process, making it more efficient and accurate. For example, when dealing with elderly patients, time-saving measures like voice dictation significantly reduce the assessment duration, leading to faster interventions and improved care.

Inpatient Care (12): Cogniwound plays a vital role in inpatient care, particularly in the management of pressure ulcers, hospital-acquired pressure ulcers, and wound documentation. Before patient admission, clinicians can use Cogniwound to document wounds accurately, ensuring that they have a complete and standardized record of the patient's condition upon entry. In cases where patients develop wounds during their inpatient stay, Cogniwound remains invaluable. The app allows real-time documentation of these wounds, ensuring that there are no delays in treatment initiation. By providing a consistent approach to wound documentation, Cogniwound minimizes variations between different clinicians and maintains high data quality. For example, when a patient transfers from one unit to another within a hospital, the software ensures that the wound assessment process remains standardized, making it easier for healthcare providers to understand the patient's condition.

Home Care (14): For home care scenarios, clinical users visiting patients can leverage the Cogniwound app to capture wound details and characteristics. This application enhances the quality of care provided at patients' homes. Through the app, healthcare providers can capture and maintain comprehensive wound data in a patient's home environment, offering the convenience and comfort of in-home care while ensuring that the patient's condition is accurately monitored. Consider a scenario where a home care nurse is attending to an elderly patient with a chronic wound. The nurse uses Cogniwound to capture wound details such as dimensions and exudate characteristics. This data is securely stored and can be easily accessed for future assessments. The app's offline capabilities further enhance home care. Even in areas with limited internet connectivity, clinical users can effectively document and later upload the data when online. This ensures continuous care for patients, even in remote locations where internet access may be unreliable.

Remote Care (16): Cogniwound extends its capabilities to remote care, allowing patients and caregivers to remotely capture wound details. This feature facilitates healthcare access for individuals who may face mobility challenges or reside in remote areas. For example, a patient living in a rural area with limited access to healthcare facilities may develop a wound. In such cases, the patient or a caregiver can use the Cogniwound app to capture detailed information about the wound. This data is securely transmitted to healthcare professionals, enabling them to assess the wound's condition remotely. The software offers secure telehealth capabilities, providing patients with timely guidance on wound care. By expanding the reach of healthcare services to underserved populations, Cogniwound ensures that patients receive the care they need, even when in-person visits are challenging or impossible.

### Usage Scenarios

Cogniwound is crafted in a way to be used as a standalone application or used as an application that seamlessly integrates with EMRs. Supporting both standalone and EMR integration scenarios is essential to cater to diverse healthcare needs and seamlessly adapt to changing requirements. While standalone use empowers healthcare providers in settings where immediate EMR access is not feasible, EMR integration offers a seamless approach for facilities with established electronic health record systems.

The ability to switch between these scenarios is a testament to Cogniwound's flexibility. Healthcare providers can transition from standalone use to EMR integration with minimal effort, ensuring that data captured in one setting can seamlessly become part of the patient's comprehensive medical record when needed. Conversely, when patients move between care settings, such as from a home care environment to an inpatient facility, healthcare providers can swiftly switch between standalone and EMR-integrated modes as the situation demands. This agility allows for a patient's wound care history to travel with them, maintaining continuity of care and enhancing overall patient outcomes. In summary, Cogniwound's support for both scenarios ensures adaptability to diverse healthcare settings and facilitates a seamless transition between them, contributing to improved patient care and efficient clinical operations.

### Standalone Usage

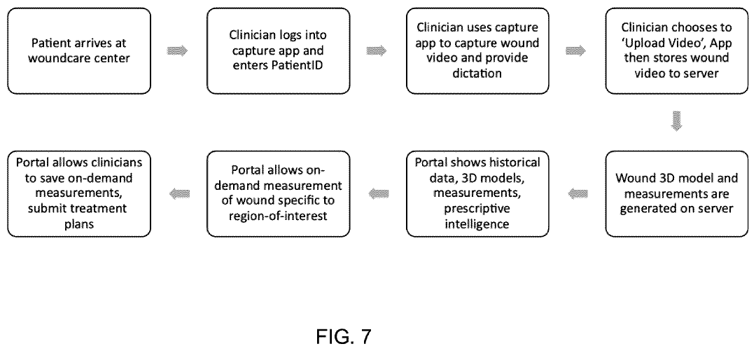

FIG. 7 depicts a flow diagram illustrating a method according to an embodiment. Specifically, FIG. 7 depicts a flow diagram of a method of using the Cogniwound system in, illustratively, a standalone usage scenario.

In this scenario, Cogniwound operates as a standalone system, providing comprehensive wound care capabilities independently. Healthcare providers use the Cogniwound mobile app and web portal to capture wound data, generate 3D models, assess wound characteristics, and access predictive and prescriptive intelligence. This autonomous setup is particularly valuable in remote or home care settings where immediate access to Electronic Medical Records (EMRs) may not be available. It ensures that users can efficiently provide wound care without relying on external systems, making it a versatile solution for various healthcare environments.

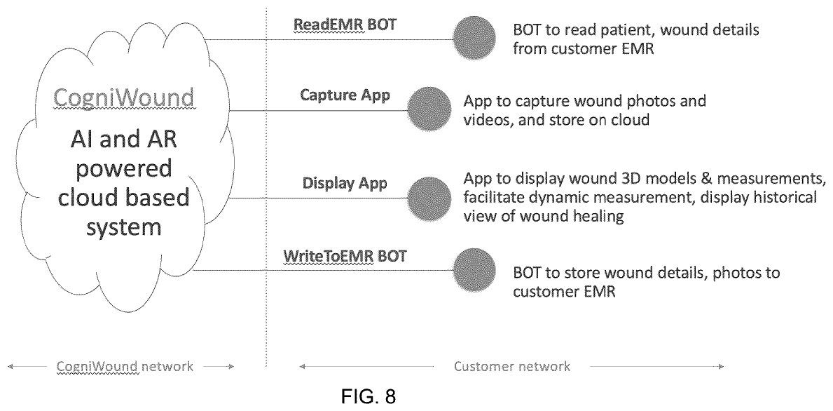

FIG. 8 graphically illustrates Cogniwound integration with EMR: Cogniwound seamlessly integrates with Electronic Medical Records (EMRs) in this scenario, enhancing the continuity of care and data management. BOTs will be created to read/write data from/to EMRs.

### Healthcare Facility

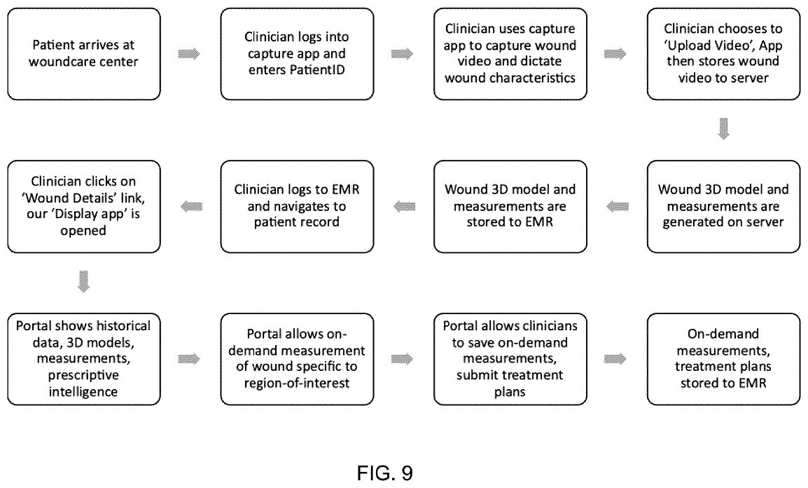

FIG. 9 depicts a flow diagram illustrating an embodiment. Specifically, FIG. 9 depicts a flow diagram of a method of using the Cogniwound system in, illustratively, a scenario whereby a patient is admitted to a healthcare facility.

When a patient is admitted to a healthcare facility, the software populates the patient's EMR with precise wound data, ensuring that the entire care team has access to up-to-date information. Wound assessments performed with Cogniwound are directly incorporated into the patient's medical record, reducing the risk of transcription errors, and streamlining the documentation process. This integration optimizes care coordination, supports data-driven decision-making, and contributes to a higher quality of care, especially in inpatient and clinic settings where EMRs are integral to healthcare operations.

### Multi-Client Setup

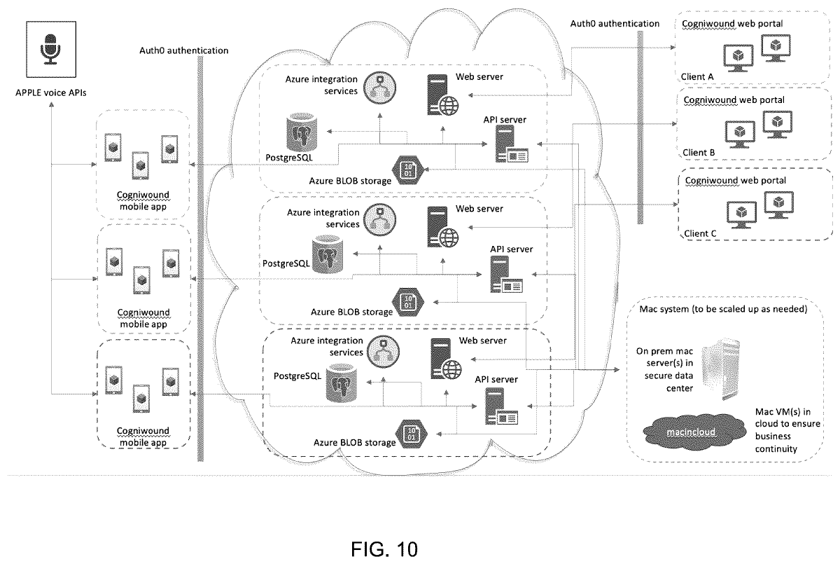

FIG. 10 depicts a high-level block diagram illustrating various functions and interactions between components of a wound assessment system according to an embodiment. Specifically, FIG. 10 depicts an embodiments such as depicted with respect to FIG. 2 wherein a multi-client setup is provided.

Cogniwound's robust architecture is designed to efficiently serve multiple clients in parallel, each with its own dedicated setup. Whether in a hospital, nursing home, home care agency, or remote care provider, Cogniwound ensures a seamless experience tailored to each client's unique needs.

In a multi-client environment, the software can be simultaneously accessed through dedicated mobile apps on individual devices. This means that nurses, clinicians, or caregivers attending to patients under different clients can install and use the Cogniwound mobile app on their respective smartphones or tablets. Each app instance is securely configured to access the specific client's data and settings, ensuring data isolation and confidentiality.

Web portals for clients offer an additional layer of accessibility and control. These web-based platforms enable administrators and healthcare providers to oversee and manage wound care for their patients. The web portals are configured with client-specific user accounts and permissions, guaranteeing that the right personnel can access and contribute to patient records as required.

The heart of Cogniwound's multi-client capability lies in its cloud-based infrastructure. Each client enjoys a dedicated setup within the cloud environment, ensuring data segregation, security, and scalability. These dedicated cloud instances can be tailored to meet the unique demands and preferences of each client, accommodating variations in data storage, processing capacity, and accessibility.

For example, in a hospital with multiple departments, Cogniwound's multi-client functionality allows each department to have its own customized instance. In this scenario, the wound care team within the surgery department can manage their patient data independently from the dermatology department, even though they are part of the same hospital network. Each department's data is kept separate, promoting efficient organization and data management.

In the context of a home care agency, where different caregivers serve patients under different clients, Cogniwound's parallel capabilities are invaluable. Each caregiver can securely access the software through a dedicated mobile app, ensuring that they collect and document wound data for their respective clients without any overlap or confusion.

Cogniwound's ability to serve multiple clients in parallel is a testament to its flexibility and scalability. It caters to diverse healthcare settings, enabling efficient wound care management, maintaining data security, and providing a tailored experience for each client's unique requirements. This multi-client approach ensures that the software can adapt to the varied demands of the modern healthcare landscape while maintaining the highest standards of data integrity and confidentiality.

### Exemplary Hardware Implementations

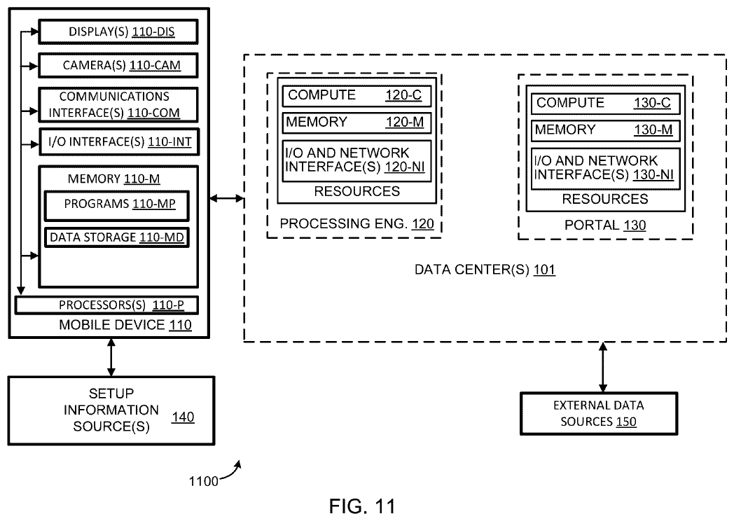

Brief descriptions of exemplary hardware implementations of several components of the Cogniwound systems previously discussed will now be described with respect to specific hardware and/or computing devices 1100 as provided in FIG. 11. Other implementations are contemplated.

Referring to the system 1100 of FIG. 11, the mobile devices 110 may comprise mobile phones, laptops, or special purpose mobile telecommunications/computing devices configured to execute applications or apps so as to perform the various functions described herein. As depicted in FIG. 11, an exemplary mobile device 110 comprises a mobile phone or other computing device having one or more processors 110-P, a memory 110-M, a input/output (e.g., user input device such as touchscreen, etc.) 110-IO, communications interface(s) (e.g., mobile network, WiFi, Bluetooth, etc.) 110-COM, one or more cameras 110-CAM, one or more displays (e.g., touchscreen display, presentation device driver, and the like) 110-DIS, and (optionally) other modules (not shown). The processor(s) 110-M are coupled to each of memory 110-M, I/O interfaces 110-INT, drivers 110-DR, and cameras 110-CAM. The processor(s) 110-P are configured for controlling the operation of mobile device 110, including operations supporting the methodologies described herein with respect to the various embodiments. Similarly, the memory 110-M is configured for storing information suitable for use by the processor(s) 110-P. Specifically, memory 110-M may store programs 110-MP, data 110-MD and so on. Within the context of the various embodiments, the programs 110-MP and data 110-MD may vary depending upon the specific functions implemented by the mobile device 110, such as setup functions, wound capture functions, secure communications functions, online and offline processing functions, interactions with the RPE 120, interactions with the portal 130, and so on as will be discussed in more detail below. Generally speaking, the mobile device may be configured with any type of hardware or combination of hardware and software suitable for use in implementing the various mobile device functions.

Referring to FIG. 11, the remote processing engine (RPE) 120 may be implemented via, illustratively, one or more data centers 101 comprising compute resources 120-C (e.g., processing resources such as provided by one or more servers, processors and/or virtualized processing elements or other compute resources), memory resources 120-M (e.g., non-transitory memory resources such as one or more storage devices, memories and/or virtualized memory elements or storage resources), input/output (I/O) and network interface resources 120-NI, and other hardware resources and/or combined hardware and software resources suitable for use in implementing a the various functions described herein with respect to the RPE 120. It is noted that various other types of virtualized services platforms, servers, and other known systems may be used to implement the RPE elements and functions such as described herein. The compute or processing resources may also be configured to execute software instructions stored in the non-transitory memory resources to provide thereby other functions as described herein.

As depicted in FIG. 11, the compute 120-C, memory 120-M, I/O and network interface 120-NI and other resources (not shown) of the data center(s) 101 are used to instantiate some or all of the Cogniwound processing engine functions described in more detail below with respect to the various figures. It is noted that while FIG. 11 depicts an architecture using virtualized RPE elements the various embodiments may also be use within the context of non-virtualized RPE elements and/or a combination of virtualized RPE elements and non-virtualized network RPE elements.

Referring to FIG. 11, the portal 130 may also be implemented via one or more data centers 101 using respective virtualized portal elements, non-virtualized portal elements, and/or a combination of virtualized portal elements and non-virtualized network portal elements in a manner similar to that described above with respect to the RPE 120.

It should be noted that aspects of the present invention may be implemented in hardware and/or in a combination of hardware and software in the mobile device 110, the processing element 120, the portal 130, and so on such as by using application specific integrated circuits (ASIC), a general-purpose computer or any other hardware equivalents. In one embodiment, such as a computer-implemented embodiment, computer instructions or code representing the various processes can be loaded into memory 904 and executed by processor 902 to implement the functions as discussed above. As such the processes (including associated data) of the present invention can be stored on a computer readable medium or carrier, e.g., RAM memory, magnetic or optical drive, server, and the like.

It is contemplated that some of the steps discussed herein as methods or processes or software methods may be implemented within computing hardware, for example, as circuitry that cooperates with the processor to perform various method steps. Portions of the functions/elements described herein may be implemented as a computer program product wherein computer instructions, when processed by a computer, adapt the operation of the computer such that the methods and/or techniques described herein are invoked or otherwise provided. Instructions for invoking the inventive methods may be stored in fixed or removable media, transmitted via a data stream in a broadcast or other signal bearing medium, and/or stored within a memory within a computing device operating according to the instructions.

### Technical Details of Exemplary Cogniwound Implementation

The following technical details are provided as a non-limiting example of how some portions of the system may be implemented. Other implementations are also contemplated.

### Cogniwound Mobile App Technical Details

The Cogniwound mobile app (18) is a versatile tool designed to operate seamlessly in a variety of care provider settings, catering to the needs of clinicians, nurses, general practitioners, surgeons, physical therapists, and more. It delivers its functionality based on advanced AI and AR capabilities, and it primarily runs on high-end smartphones, such as the iPhone Pro series models.

The Cogniwound app (18) may be deployed on AI and AR capable smartphone (in the case of iPhones, these would be the pro series model iPhones)

In an out-patient setup (10) Cogniwound app (18) would be used by nurses and/or clinicians, during scheduled patient visits to capture wound photos and/or wound videos, dictate wound characteristics.

In an in-patient setup (12) Cogniwound app (18) would be used for treating pressure ulcers and hospital acquired pressure ulcers. Cogniwound can also be used at nursing homes as well as Long-term acute care hospitals (LTACHs). Cogniwound provides the ability to document a wound, prior to a patient being admitted. If patient develops a wound during their treatment at the centers, Cogniwound can be used for documenting that aspect too.

In homecare setup (14), the clinical user visiting the patient uses Cogniwound app (18) to capture wound details and characteristics

In a remote care setup (16), the patient/caregiver uses Cogniwound app (18) to capture wound details.

Wound capture (22) uses advanced AI techniques to identify the optimal number of 2D wound photos and/or optimal length wound videos to create a 3D model. It will auto-position mobile camera to capture wound photos and/or wound videos from various perspectives and angles.

Wound characteristics dictation (22) module uses Apple Inc's Voice APIs (24) to capture voice responses to wound characteristics questions, converts them to text and stores them in the database (60) Wound characteristics questions are a set of structured questions developed by Cogniwound SMEs to accurately diagnose wounds and identify treatment plans.

In one embodiment of the Cogniwound app (18), the inventors used the following libraries/functions/packages/utilities/technologies/software: SwiftUI; UlKit; Alamofire; Auth0; SimpleKeychain; RxSwift; JWTDecode; Model3DView, SceneKit; AlertToast; AVFoundation; Speech; CoreGraphics; CoreImage; Network; CoreMotion; CoreData; and AVFoundation.

### Cogniwound Engine Technical Details

Cogniwound engine (50) is a cloud based central processing engine comprising of multiple modules that work together to create the 3D modelling, wound measurement, wound diagnosis, predictive and prescriptive intelligence, and a self-learning context-aware wound care knowledgebase.

Cogniwound engine (50) has the API services (55) that connect to various modules across all three components-Cogniwound app (18), Cogniwound engine (50) and Cogniwound web portal (100).

Database (60) stores information related to wound, patient, patient encounter, treatment plans and wound knowledgebase.

Cogniwound app (18) sends wound photos, wound videos, and wound characteristics to Cogniwound engine (50) in chunks and these get stored in file/BLOB storage (65). This will result in faster data transfer from the Cogniwound app (18) to the Cogniwound engine (50). The chances of memory leaks and memory crashes within Cogniwound app (18) are drastically reduced by transmitting the data in chunks. API services (55) convert chunked data in file/BLOB storage (65) and store them in database (60).

In one embodiment of the API services (55), the inventors used the following libraries/functions/packages/utilities/technologies/software: Express; Cors; express-oauth2-jwt-bearer; azure storage-file-share; adm-zip; axios; base64-stream; compression; dotenv; etag; heic-convert; multer; multiparty; postgress; typescript; uuid; nodemon; and npm-run-all.

2D wound data captured from wound photos and/or wound videos by Wound capture (20) is sent to Mac processor (70) by Cogniwound engine (50), Mac processor (70) converts this 2D wound data into 3D model and stores them in database (60). Mac processor (70) uses Apple's RealityKit Object Creation utilities to generate 3D models based on the 2D wound data

In one embodiment of the Mac processor (70), the inventors used the following libraries/functions/packages/utilities/technologies/software: Foundation; os; RealityKit; Metal; express; postgress; typescript; nodemon; exec-sh; sub-process; dotenv; and cors.

Web portal (75) hosts the needed web pages to display wound information, 3D models, wound measurement, custom measurement, and wound care knowledgebase

Integration services (80) act as interfaces between Cogniwound and external systems. They send/receive data to/from external systems viz. Patient data (105), Wound data (110), Data from EMRs (115), Other data (120) or data from other systems.

Knowledgebase (85) a.k.a wound care knowledgebase is the core intelligence unit of Cogniwound. This consists of natural language processing (NLP) based machine learning systems that continually ingest wound data, patient data, treatment plans to learn from additions or updates to clinical procedures and/or treatment plans. Pre-trained models like BERT, Med-BERT, ClinicalBERT, T5, XLNet are used by Knowledgebase (85).

P&P intelligence (90) module provides wound healing trend predictive and wound healing prescriptive intelligence. It does this by employing advanced AI and ML algorithms, ensembling methods, that read from wound care knowledge base (85) and combine it with wound healing trends, patient wound history, historical information of similar wounds and/or patients treated.

P&P intelligence (90) module leverages U-Net, DeepLab, and Mask R-CNN segmentation models to detect wound objects (or wound region-of-interest) within wound images.

P&P intelligence (90) module leverages Convolutional Neural Networks (CNNs) to classify different wound types (e.g., pressure ulcers, diabetic foot ulcers) based on the characteristics of the wound object detected.

P&P intelligence (90) module leverages Multi-Task Learning (MTL) models to perform wound classification, wound area estimation, healing prediction, wound risk assessment and treatment recommendation simultaneously

Continual learning (95) module leverages Elastic Weight Consolidation (EWC), Gradient Episodic Memory (GEM), Incremental Classifier and Representation Learning (iCaRL), Continual Conditional GAN (CCGAN) models to identify the differences between the treatments prescribed and the treatments administered to determine the variations in the treatment plans and correlate them to wound and/or patient specific conditions. The correlated information is then fed back to the wound care knowledgebase (85).

Generative Intelligence (97) module leverages Generative Adversarial Network (GAN) models to generate new wound images based on a smaller set of baselined wound images.

### Cogniwound Portal Technical Details

Cogniwound portal (100) is a web-based system comprising of multiple modules that work together to render/deliver 3D modelling, wound measurement, wound diagnosis, predictive and prescriptive intelligence, and a self-learning context-aware wound care knowledgebase.

Cogniwound portal (100) uses API services (55) that connect to various modules across Cogniwound engine (50) to provide the necessary views/pages to users based on their roles and privileges.

Cogniwound portal (100) provides views on wound measurements, wound 3D models, wound care knowledge base, predictive and prescriptive intelligence.

In one embodiment of the Web portal (75) and Cogniwound portal (100), the inventors used the following libraries/functions/packages/utilities/technologies/software: angular; Auth0; angular-fontawesome; fontawesome-svg-core; free-brands-svg-icons; free-regular-svg-icons; free-solid-svg-icons; echarts; webxr; echarts; echarts-gl; fabric; heic-convert; install; lil-gui; meshline; ngx-custom-validators; ngx-echarts; ngx-editor; ngx-spinner; ngx-toastr; npm; rxjs; three; three.meshline; threejs-slice-geometry; zone.js; bootstrap; axios; and tslib.

### Cogniwound Platform Technology Components

This section provides details on the various components within the platform. These components collectively deliver system functionality.

### User Management

Auth0 Identify Access Management platform is used to authenticate and authorize platform users. The authorization is setup across mobile app, API, portal, and user management screens. User and User_roles tables have been created to use Auth0 APIs and deliver needed functionality. Passwords are not stored in Cogniwound database.

### Users-Roles

There are four general user roles within the platform-super admin, administrator, doctor, and nurse. The super admin role will be specific to Cogniwound support team. This role will be used to create one or more administrator accounts (admin users) for a client. Admin users of a client will be able to add users and assign doctor or nurse roles. Users with doctor role will be able to ‘submit’ patient visit records

### Mobile App

This is developed using SWIFT UI and Model-view-viewmodel (MVVM) architecture. Reactive UI is used as part of voice-based characteristics documentation, to dynamically change UI based on the responses received from the user. Wound images will be captured in *.HEIC format and corresponding meta data will be captured as *_gravity.TIF and *_depth.TXT files.

### Cloud

Azure based cloud system is used for development and hosting various components-APIs, DB, hosting the portal, send/receive data from Mac processor (or MAC APIs). Azure database for PostgreSQL is used as the database General purpose storage (StorageV2) is used for storing files/BLOBs

### APIs

REST framework is used to build Cogniwound system APIs, using TypeScript and Node.js. All the APIs are hosted on Azure. These APIs can be called from either the mobile app, or the Mac processor or from portal client. The following APIs of Table 1 are an indicative list of current APIs:

TABLE 1

create_entry.ts
create_patient.ts
create_user.ts
delete_user.ts
files_upload.ts
get_all_files_in_session.ts
get_all_patients_offline.ts
get_all_patients.ts
get_auth_token.ts
get_model_session.ts
get_model.ts
get_notes.ts
get_patient.ts
get_question_and_answers.ts
get_questions.ts
get_session_measurements.ts
get_thumbnails.ts
get_transcript.ts
get_user.ts
post_doc_answers.ts
roles.ts
save_measurements.ts
save_notes.ts
search.ts
session_submit.ts
upload_status.ts

The following interfaces/functions are defined to be used within the APIs:
Patient_data.ts—Patient interface structure
answers.ts—answers (voice dictation) interface structure
db.ts—functions to get data for various screens/pages
measurements.ts—Measurements interface structure
question.ts—question interface structure
response.ts—interface definition and retrieve response function
status.ts—interface definition and retrieve status function
upload_status.ts—upload_status interface structure

### Database

PostgreSQL database is used as the backend. The following Table 2 is an indicative list of tables currently used:

TABLE 2

| Table | Description |
|---|---|
| public.ethnicity | Ethnicity domain table |
| public.gender | Gender domain table |
| public.image_uploads | stores images related data for every |
| | patient session |
| public.lookup_status | stores patient session status |
| public.lookup_upload_status | lookup table for upload status |
| | of a patient session to cloud |
| public.medical_history | stores patient medical history |
| public.patient | stores patient details |
| public.patient_sessions | stores patient session details |
| patient_sessions_offline | stores patient session details |
| | captured in offline mode |
| public.session_files | stores details of files associated |
| | with a patient session |
| public.video_upload | stores details of file uploads |
| public.wound_description | table for storing word |
| | characteristics |
| public.wound_doc_question_op- | stores types of questions that |
| tions | can be asked during dictation |
| public.wound_doc_questions | stores questions for use during |
| | voice dictation |
| public.wound_doc_transcript | stores responses received |
| public.wound_documentation_ques- | stores questions that will be |
| tions | displayed in portal |
| public.wound_sessions | stores details of wound sessions |

### File System.

File and BLOB storage on Azure is used to store 2D images, their meta data, and the generated 3D models.
cw-images-model-files is the root folder under which each patient sessions are shown as subfolders,
   /cw-images-model-files/026ef8cc-01a7-46b1-ab7a-1683014a7d02 is a subfolder for patient session 026ef8cc-01a7-46b1-ab7a-1683014a7d02
   This subfolder will have 2 subfolders further-source and generated
   Source subfolder
      Will have an images subfolder in it which stores the images and metadata
      /cw-images-model-files/026ef8cc-01a7-46b1-ab7a-1683014a7d02/source/images
      For example, for Patient ID, 9885810CG, and first image capture (IMG_0001) the following 3 files will be stored in the folder
         9885810CG_IMG_0001.HEIC—image file
         9885810CG_IMG_0001_depth.TIF—depth related information
         9885810CG_IMG_0001_gravity.TXT—gravity meta data
      Approximately 20-40 images are generated per patient session. The actual number of images depends on the operating conditions when the wound video was captured (lighting, intensity, proximity to wound etc)
   Generated subfolder
      Will have the 3D models generated (models subfolder) and thumbnails (thumbnails subfolder) to be shown for a 3D model
      For example, /cw-images-model-files/026ef8cc-01a7-46b1-ab7a-1683014a7d02/generated/models has the following files
         baked_mesh.mtl—Material Library File (.mtl) containing one or more material definitions, each of which includes the color, texture, and reflection map of each wound image. These are applied to the surfaces and vertices of objects.
         baked_mesh.obj—contains information about the geometry of 3D objects
         baked_mesh.usda—scene description files
         baked_mesh_ao0.png—point in time image file
         baked_mesh_norm0.png—point in time image file
         baked_mesh_tex0.png—point in time image file
         log_1.txt—informational log
      /cw-images-model-files/026ef8cc-01a7-46b1-ab7a-1683014a7d02/generated/thumbnails will have the image to be shown as a thumbnail

### MAC Processor

Mac processor is used to generate 3D models from the 2D wound images and meta data captured by the mobile app. Mac processor keeps polling the database and checks for any new entries in patient session table. Once it finds a new entry, the session details are pulled into the mac processor and 3D model generated. Photogrammetry library and iOS libraries are used when generating the 3D model. Once 3D model is generated, the details are sent back to the DB and stored.

### Portal Client

Portal client is built using Angular framework. 3D models are displayed using three.js framework. Data is retrieved from database using API calls. All API calls are authenticated using Auth0.

### Cogniwound Code Snippets

Provided below are some illustrative code snippets from the Cogniwound codebase.

```
Pre-processing
from transformers import BertTokenizer
# Load tokenizer
tokenizer = BertTokenizer.from_pretrained(‘bert-base-uncased’)
# Example for a single treatment plan
text = “The patient presented with a deep wound on the leg. Recommended treatment includes
cleaning the wound, applying antibiotic ointment, and dressing it daily.”
encoded_text = tokenizer.encode_plus(
  text,
  add_special_tokens=True,
  max_length=128,
  pad_to_max_length=True,
  return_attention_mask=True,
  return_tensors=‘pt’
)
input_ids = encoded_text[‘input_ids’]
attention_mask = encoded_text[‘attention_mask’]
Fine-tuning
import torch
from transformers import BertForSequenceClassification, AdamW
# Load pre-trained model with a classification head
model = BertForSequenceClassification.from_pretrained(‘bert-base-uncased’,
num_labels=num_classes)
# Define optimizer and loss function
optimizer = AdamW(model.parameters( ), lr=2e−5)
loss_fn = torch.nn.CrossEntropyLoss( )
# Fine-tuning loop
for epoch in range(num_epochs):
  model.train( )
  for batch in dataloader: # Iterate through your dataset batches
    input_ids, attention_mask, labels = batch
    optimizer.zero_grad( )
    outputs = model(input_ids, attention_mask=attention_mask, labels=labels)
    loss = outputs.loss
    loss.backward( )
    optimizer.step( )
Model Evaluation
model.eval( )
total_correct = 0
total_samples = 0
with torch.no_grad( ):
  for batch in test_dataloader: # Iterate through your test dataset batches
    input_ids, attention_mask, labels = batch
    outputs = model(input_ids, attention_mask=attention_mask)
    _, predicted_labels = torch.max(outputs.logits, dim=1)
    total_correct += (predicted_labels == labels).sum( ).item( )
    total_samples += len(labels)
accuracy = total_correct / total_samples
print(f“Test Accuracy: {accuracy}”)
Image classification
import dependencies
# Image data directories
train_dir = “path/to/train/directory”
validation_dir = “path/to/validation/directory”
# Data augmentation
train_datagen = ImageDataGenerator(
  rescale=1.0 / 255.0,
  rotation_range=20,
  width_shift_range=0.2,
  height_shift_range=0.2,
  shear_range=0.2,
  zoom_range=0.2,
  horizontal_flip=True,
  fill_mode=“nearest”
)
validation_datagen = ImageDataGenerator(rescale=1.0 / 255.0)
img_size = (224, 224)
batch_size = 32
# Create and validate model
# .....
# code intentionally not shown
# .....
# Train the model
num_epochs = 10
model.fit(train_generator, epochs=num_epochs, validation_data=validation_generator)
# Save the model
model.save(‘wound_classification_model.h5’)
```

### Benefits and Usefulness

Cogniwound revolutionizes wound care with advanced AI and 3D modeling, offering precise measurements and timely interventions. It provides personalized recommendations, integrates seamlessly with EMRs, and adapts to both online and offline scenarios. By enabling remote care and reducing SME dependency, Cogniwound ensures comprehensive, efficient, and continually improving wound assessment without the need for external medical devices.

Enhanced Precision: Cogniwound offers a significant advantage over manual wound measurements. It uses advanced AI and 3D modeling to precisely measure wound dimensions. For example, when assessing a patient with chronic pressure ulcers, traditional manual measurements may be subject to human error. In contrast, Cogniwound ensures highly accurate measurements by capturing the wound's true dimensions, leading to more reliable data for diagnosis and treatment planning.

Time Efficiency: The software significantly reduces the time needed for wound assessment. Consider a busy hospital setting where a clinician has multiple patients with various wound types. Cogniwound streamlines the process, allowing the clinician to capture wound data rapidly and accurately. This speed ensures timely interventions and minimizes the risk of complications.

3D Wound Models: Cogniwound's 3D wound models outshine traditional 2D photographs. In the case of a patient with a complex surgical wound, the ability to view and manipulate a 3D model offers superior insights. Surgeons can precisely assess wound depth, evaluate tissue health, and plan interventions more effectively than using conventional 2D images.

Predictive and Prescriptive Intelligence: Cogniwound's AI-driven predictive and prescriptive intelligence provides a notable advantage in wound care. Let's consider a scenario where a clinician must determine the optimal treatment for a patient with a diabetic foot ulcer. Cogniwound analyzes historical patient data and wound characteristics to offer personalized recommendations. This feature increases the likelihood of successful treatment and better patient outcomes.

Integration with EMRs: Cogniwound's seamless integration with Electronic Medical Records (EMRs) sets it apart. When compared to standalone wound care documentation tools, the software ensures consistent and error-free data transfer. For instance, when admitting a patient to a hospital with an existing wound, Cogniwound directly populates the patient's EMR with accurate wound data. This integration saves time, reduces the risk of transcription errors, and ensures that all healthcare providers have access to the most up-to-date information.

Versatile Deployment: The software's adaptability is a key advantage. Let's take the example of a home care nurse visiting an elderly patient. Cogniwound's offline mode enables the nurse to document the wound and characteristics, even in areas with limited internet connectivity. This flexibility ensures that wound care can be provided without interruptions, resulting in better outcomes for the patient.

Offline and Online Modes: Cogniwound's ability to work in both offline and online modes gives it a distinct edge over solutions that solely rely on an internet connection. In remote areas with unreliable connectivity, patients and clinicians can still capture and store wound data offline. Later, when online, the data is securely transmitted to the central database. This flexibility is especially advantageous for ensuring continuous wound monitoring and treatment.

Structured Wound Assessment: Cogniwound provides structured wound assessment, ensuring a standardized approach to evaluating wound conditions. Traditional methods may vary in the way wound assessments are conducted, leading to inconsistencies. With structured assessment, healthcare providers have a common framework for evaluating wounds. For instance, when assessing wound exudate, Cogniwound offers standardized questions like “Is exudate present?” or “Exudate amount?” This structured approach results in more accurate data and consistent diagnosis and treatment planning.

Less Scope of Infection as there is no Annual Wound Measurement: Cogniwound's continuous wound monitoring reduces the risk of infections. In traditional wound care, annual measurements can lead to prolonged periods between assessments, increasing the potential for infections to go unnoticed. Cogniwound's ability to provide real-time data and alert healthcare providers to changes in wound conditions minimizes the scope for infections. For example, when tracking a patient's surgical wound, if there is an unusual increase in exudate, Cogniwound can promptly notify the clinician to act.

Model's Continual Learning Based on the Treatments Administered: Cogniwound employs continual learning to enhance its wound care knowledge base. When treatments are administered, the software tracks the outcomes and their effectiveness. For instance, if a certain treatment plan leads to rapid wound healing in patients with similar conditions, Cogniwound learns from this data and can recommend the same treatment for other patients with similar profiles. This continual learning ensures that the software evolves and becomes more effective over time.

Personalized Wound Care: Cogniwound stands out by offering personalized wound care recommendations. It tailors its suggestions based on the specific needs and characteristics of each patient. For example, if two patients with diabetic foot ulcers have distinct wound characteristics and medical histories, Cogniwound would recommend different treatments. This personalized approach increases the likelihood of successful outcomes and a more patient-centered experience

Enabling Remote Care: Cogniwound empowers healthcare providers to offer wound care remotely, expanding the reach of medical services. In scenarios where patients cannot easily access healthcare facilities, such as rural areas or during a global pandemic, remote care becomes crucial. With Cogniwound, patients can capture wound data at home using the mobile app. The software allows healthcare providers to remotely assess wounds, monitor progress, and offer timely guidance. For example, a homebound elderly patient with a chronic wound can use Cogniwound to capture data, and a wound specialist can remotely view the wound, assess its condition, and recommend treatment adjustments. This approach reduces the need for in-person visits, ensuring the patient receives necessary care without leaving their home.

Less Dependency on Subject Matter Experts (SMEs): Cogniwound minimizes the reliance on specialized expertise to diagnose and treat wounds. Traditional wound care often necessitates the presence of SMEs, such as wound care specialists, to assess and make recommendations. With Cogniwound's AI-driven features, even healthcare providers who are not wound care specialists can confidently assess wounds and make informed decisions. For example, in a busy outpatient clinic, a general practitioner can use Cogniwound to assess wounds with confidence, knowing that the software's intelligence and knowledge base provide accurate insights and recommendations. This reduction in SME dependency streamlines the care process and improves access to expertise.

Model Becoming Smarter Day-by-Day: Cogniwound's AI and machine learning capabilities allow the model to evolve and improve continually. As more patients are treated and more data is gathered, the software refines its wound care knowledge. For instance, when multiple patients with similar wound conditions receive different treatment plans, Cogniwound learns from the treatments administered. Over time, the software recognizes the most effective approaches for specific wound types and patient profiles. This continual improvement ensures that Cogniwound adapts to changing best practices, ultimately resulting in better wound care outcomes.

No External Medical Devices Needed to Measure: Cogniwound simplifies the wound assessment process by eliminating the need for additional medical devices. Traditional wound measurement techniques might require separate instruments, such as rulers and specialized cameras. Cogniwound's mobile app serves as a comprehensive tool, allowing users to capture wound data without relying on external devices. For example, when capturing a wound, the app utilizes AI to automatically calculate wound dimensions, eliminating the need for manual measurements. This self-contained approach minimizes the complexity of wound assessment and reduces the likelihood of measurement errors.

Knowledgebase: Cogniwound's Knowledgebase enhances wound care with its vast database of wound care protocols, treatment procedures, and continually updated clinical insights. Healthcare providers benefit from standardized, data-driven decisions and up-to-date guidelines. For instance, when diagnosing a complex wound, the Knowledgebase ensures that clinicians have access to the latest research and best practices, leading to more informed treatment plans. This wealth of knowledge minimizes errors and inconsistencies in wound care, ultimately improving patient outcomes.

### AI and AR Usage

In Cogniwound, AI (Artificial Intelligence) and AR (Augmented Reality) are integral components used across various stages of wound care, revolutionizing the way healthcare providers diagnose and treat wounds.

Auto-Position Phone Camera and Capture Optimal Wound Views: AI-driven algorithms automatically position the phone's camera to capture wound images and videos from the most advantageous perspectives. For instance, when assessing a complex surgical wound, Cogniwound ensures that every angle and detail is captured with precision, minimizing the need for manual camera adjustments.

Generate 3D Models from Wound Videos and Images: Utilizing advanced computer vision and AR technology, Cogniwound transforms 2D wound photos and videos into highly accurate 3D models. This provides a comprehensive view of the wound's structure, which is particularly valuable for deep or complex wounds.

Render Wound 3D Models on Mobile Devices: AR is used to render these 3D models directly on mobile devices, allowing clinicians to interact with and manipulate them in real-time. This enhances the visualization of wound dimensions and characteristics.

Determine Wound Dimensions: AI algorithms calculate precise wound dimensions, such as length, width, depth, circumference, area, and volume, providing accurate quantitative data for diagnosis and treatment planning. This eliminates the subjectivity and errors associated with manual measurements.

Detect Wound Characteristics: The software employs AI to automatically detect wound characteristics, including exudate, tissue health, granulation tissue, slough, and necrosis. These detections are vital for understanding the wound's condition.

Recognize Patterns in Wound Images for Different Wound Types: Cogniwound's AI utilizes pattern recognition to categorize wounds based on their visual characteristics. For instance, it can distinguish between pressure ulcers, diabetic foot ulcers, and other wound types, aiding in accurate diagnosis.

Detect and Classify Wounds Based on Characteristics: AI-based classifiers analyze wound characteristics and classify wounds into specific categories, enabling healthcare providers to quickly identify the type of wound they are dealing with. For instance, it can differentiate between an infected wound and a healing wound.

Recommend Personalized Wound Healing Plans: Cogniwound leverages AI-driven predictive and prescriptive intelligence to recommend personalized treatment plans based on the wound's specific characteristics and the patient's history. For example, it can suggest specific wound dressings, medications, or interventions based on the wound's condition.

Self-Learn Based on Administered Treatments: The software continually learns from the treatments administered to patients. When a treatment is particularly effective for a specific wound type or condition, Cogniwound adapts and incorporates this knowledge into its recommendations. This self-learning feature ensures that the software becomes more effective over time, improving wound care outcomes.

In summary, AI and AR are woven into Cogniwound's fabric, enhancing precision, automating complex tasks, improving diagnosis, and offering personalized wound care guidance. This technology-driven approach redefines wound care, making it more efficient, accurate, and patient-centered.

### Building a Knowledge Base

In a different embodiment, the system is equipped with an AI and ML-based processing engine designed to construct a comprehensive knowledge base specific to wound care. This knowledge base draws from a wide array of sources, including wound characteristics, patient attributes, patient-wound history, standardized wound care procedures and protocols, and actual treatment plans administered. Furthermore, the system is designed to interface with external systems to retrieve essential patient details, wound-specific information, and wound treatment data. A particularly noteworthy aspect of this embodiment is its capacity to continually learn from real-world wound care procedures and protocols, thereby improving the knowledge base over time.

### Connectivity and Learning

This embodiment underscores the system's interconnected nature. It showcases the system's ability to connect with other healthcare systems and databases, enabling the retrieval of vital patient information, wound-specific details, and wound treatment histories. The system's overarching objective is to continuously learn from actual wound care procedures and protocols administered, leveraging this knowledge to enhance its predictive and prescriptive capabilities.

### AI and AR Powered Web Portal

In one embodiment, the system includes a web portal that harnesses Artificial Intelligence and Augmented Reality technologies. The web portal serves several pivotal functions:

Rendering of 3D Models: The portal has the ability to render detailed 3D models of wounds. This feature enhances the depth and accuracy of wound analysis.

Measurement on 3D Models: The portal facilitates precise measurements on these 3D models, an invaluable resource for healthcare professionals in tailoring treatment plans.

Predictive Insights: The web portal offers predictive views of wound healing trends and timelines. This predictive capability is underpinned by advanced statistical and mathematical models.

Prescriptive Intelligence: The portal provides prescriptive intelligence, offering actionable recommendations for wound healing based on data analysis. This sophisticated feature not only expedites treatment planning but also enhances the overall quality of care provided to patients.

The following components are described herein with respect to some or all of the various embodiments:
1. A component to perform no-touch measurements using advanced algorithms built into Augmented Reality capable smartphones
2. A component to determine wound metadata (gravity, depth, and alpha mask) at the time of wound 2D image capture
3. A component to identify optimal number of 2D wound photos to build a wound 3D model
4. A component to build wound 3D models
5. A component to display wound 3D models in Augmented Reality capable smartphones
6. A component to display wound 3D model in web browsers
7. A component to auto position smartphone camera to capture wound photos and/or wound videos from various viewpoints
8. A component to gather, collate, merge, integrate and standardize wound care protocols and procedures
9. A voice-interactive component to enable wound characteristics dictation. This includes voice to text conversion and natural language processing (NLP) capabilities
10. A component to capture the observables data in and around the wound vicinity
11. A computer processor configured to execute AI & ML methods to auto determine wound measurements (width, height, depth, area, circumference, and volume).
12. A computer processor configured to display identified actual parameters like depth, area, etc., in a graphical display unit.
13. A computer processor configured to validate the calculated critical parameters via user interface.
14. A computer processor to convert 2D wound data (wound images and associated metadata, including gravity, depth, and alpha mask) to 3D wound models.
15. A component to enable precise custom measurements on wound 3D models
16. An AI and ML based component to auto-detect wound object i.e., region-of-interest (RoI) from wound images
17. An AI and ML based component to auto-classify a wound based on the characteristics of the detected wound object i.e., region-of-interest (RoI)
18. An AI and ML based component to auto-diagnose a wound based on wound classification, wound characteristics, historical wound information, patient medical history, patient demographics, patient habits, and similar wound treatment procedures and protocols
19. A computer processor configured to get the color and shape skin tissue related information to compute the criticality of the wound
20. A computer processor that integrates modules within Cogniwound and allows it to communicate with other external systems
21. AI based Ensemble models to predict the critically of wound and wound healing time
22. A prescriptive intelligence generation model to provide intelligence and recommendations on wound care to be implemented
23. An AI and ML based component to continually self-learn from actual wound treatments administered and improve knowledge base
24. A generative AI based model to generate additional wound images based on a smaller set of wound images

Cogniwound is an integrated system that is not only technologically advanced but also patient-focused. It automates the data collection process, enhances diagnosis and treatment planning, and continually improves its capabilities through learning. By doing so, it streamlines wound care procedures, reduces costs, and ultimately elevates the quality of care provided to patients, making it a pivotal tool in the domain of wound care.

As discussed above, the Cogniwound mobile app provides a comprehensive functionality ensuring that vital wound data is collected and made available for further analysis, diagnosis, and treatment planning. This data-driven approach, supported by advanced technologies, is fundamental in the quest to optimize wound care and elevate patient outcomes.

As discussed above, the Cogniwound system and methods provide an all-encompassing solution and platform that leverages an array of technical innovations to optimize wound care. It is not just an advancement; it represents a paradigm shift in how wounds are managed. Cogniwound in various embodiments advantageously: Automates the measurement and documentation of wounds, ensuring that crucial data is collected accurately and comprehensively; Facilitates collaboration among healthcare professionals, streamlining wound care procedures and protocols to deliver standardized and effective treatment; Predicts wound healing trends and prescribes treatment plans, eliminating guesswork and uncertainty in wound care; Adopts a continuous learning approach, constantly improving its predictive and prescriptive capabilities based on real-world experiences; Substantially reduces the overall cost of wound care, making it a cost-effective and efficient solution; Elevates the quality of wound care, enhancing the experiences of patients and improving patient outcomes significantly.

In essence, the Cogniwound mobile app offers a seamless and efficient process for capturing wound data, rendering 3D models, and monitoring the healing progress. Its user-centric design and advanced technology ensure that wound care providers can optimize their interactions with patients while maintaining a high standard of data accuracy and security.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof.

What is claimed is:

1. A method of wound assessment and care, comprising:
at a mobile device, capturing wound related imagery and respective audio annotation, and transmitting the wound related imagery and respective audio annotation towards a remote processing engine;
at the remote processing engine:
processing received wound related imagery to create a respective three-dimensional (3D) model of the wound;
determining wound measurements using vertices and edges defined by the 3D model of the wound;
determining wound characteristics using the wound measurements;
determining wound classification using the wound characteristics and audio annotation;
retrieving a wound care treatment for the wound classification from a knowledge base; and
transmitting the wound care treatment toward the mobile device.

2. The method of claim 1, wherein the wound imagery includes imagery depicting the wound and imagery depicting the body location of the wound.

3. The method of claim 1, wherein the audio annotation comprises first responder dictation describing any of the wound, the wound location, and tissue surrounding the wound.

4. The method of claim 1, wherein the mobile device is configured to interactively guide a user capturing the wound related imagery and respective audio annotation.

5. The method of claim 4, wherein the mobile device interactively guides a user to capture imagery of tissues surrounding and proximate the wound.

6. The method of claim 4, wherein the mobile device interactively guides a user to capture video imagery of the wound from a predefined distance from the wound and for a predefined period of time.

7. The method of claim 6, wherein the mobile device interactively guides a user to describe characteristic of the wound during the capture of the video imagery of the wound.

8. The method of claim 1, wherein the mobile device processes received audio information using a natural language processing (NLP) module invoked at the mobile device to generate thereby a textual representation of the audio information for transmission to the remote processing engine.

9. The method of claim 1, wherein the mobile device is configured to determine wound metadata including at least one of gravity, depth, and alpha mask at the time of wound 2D image capture.

10. The method of claim 1, wherein the mobile device includes an Augmented Reality capability configured to enable no-touch wound measurements.

11. A mobile device comprising a memory and a processor, the memory for storing instructions which, when executed by a processor, configure the mobile device to perform a method of capturing wound related information, the method comprising:
capturing wound related imagery and respective audio annotation; and
transmitting the wound related imagery and respective audio annotation towards a remote processing engine, the wound related imagery and respective audio annotation configured to cause the remote processing engine to responsively generate therefrom a respective three-dimensional (3D) model of the wound, determine wound measurements using vertices and edges defined by the 3D model of the wound, determine wound characteristics using the wound measurements, determine wound classification using the wound characteristics and audio annotation, retrieve a wound care treatment for the wound classification from a knowledge base; and transmit the wound care treatment toward the mobile device.

12. A cloud-based processing system comprising memory and compute resources configured to perform a method, comprising:
receiving, from a mobile device, wound related imagery and respective audio annotation;
processing the received wound related imagery to create a respective three-dimensional (3D) model of the wound;
determining wound measurements using vertices and edges defined by the 3D model of the wound;
determining wound characteristics using the wound measurements;
determining wound classification using the wound characteristics and audio annotation;
retrieving a wound care treatment for the wound classification from a knowledge base; and
transmitting the wound care treatment toward the mobile device.
